(19) United States
(12) Patent Application Publication
(10) Pub. No.: US 2005/0148986 A1
(43) Pub. Date: Jul. 7, 2005

(54) Disposable absorbent article having a dual fastening system

(76) Inventors: Janet E. Collins, Hortonville, WI (US); Barbara A. Gossen, Oshkosh, WI (US); Connie M. McMorrow, Menasha, WI (US); Thomas H. Roessler, Appleton, WI (US); Wendy L. Van Dyke, Appleton, WI (US)

Correspondence Address: KIMBERLY-CLARK WORLDWIDE, INC., 401 NORTH LAKE STREET, NEENAH, WI, 54956, US

(21) Appl. No.: 10/749,997

(22) Filed: Dec. 31, 2003

(51) Int. Cl. A61F 13/15; A61F 13/20

(52) U.S. Cl. 604/387

(57) ABSTRACT

Disclosed is a dual fastening system. The dual fastening system includes a pair of primary fasteners and a pair of secondary fasteners. Also disclosed is a disposable absorbent article including such a dual fastening system.

### BACKGROUND

Conventional disposable absorbent articles, such as disposable diapers, have typically included a bodyside liner, an outer cover and an absorbent core disposed between the outer cover and the bodyside liner. The disposable absorbent articles have generally defined a front waist region, a rear waist region and a crotch region which extends between and connects the front and rear waist regions. Such conventional disposable absorbent articles have also included fastening systems which are configured to secure the article on the wearer's waist. The disposable absorbent articles have also been constructed with various types of elasticized portions at the waist and leg opening regions. Such elasticized portions have been used to reduce the leakage of body exudates from the disposable absorbent article and improve the appearance and fit of the article about the wearer.

Typically, the fastening systems on conventional disposable absorbent articles have included a pair of fasteners located on the outermost corners of the article in one of the waist regions of the article. Such fasteners have been configured to releasably engage a complimentary fastener in the opposite waist region of the disposable absorbent article. For example the fastening systems have included a pair of fasteners, such as adhesive tape tabs, located on the outermost corners of the disposable absorbent article in the rear waist region of the article. Such systems have also included a complimentary fastener, such as a taping panel, located on the outer surface of the outer cover of the disposable absorbent article in the front waist region of the article. In such a configuration, the disposable absorbent article has been positioned between the legs of the wearer and the adhesive tape tabs have been releasably attached to the taping panel to secure the article about the waist of the wearer.

However, conventional disposable absorbent articles which are configured as described above have exhibited several shortcomings. For example, it has been difficult to maintain and conform conventional disposable absorbent articles and, in particular, the absorbent cores of such articles about the body of the wearer. It is not unusual for the absorbent cores on such conventional disposable absorbent articles to sag or droop, especially after becoming saturated with body exudates. Such sagging and drooping has undesirably resulted in improper fit and appearance which has led to increased leakage. Moreover, the outer covers on such disposable absorbent articles have exhibited a “blousy” appearance while in use.

In an attempt to better gather the absorbent core and the disposable absorbent article, the waist and leg opening regions of conventional disposable absorbent article have included elastics which apply high contracting or gathering forces on the article. However, such high forces have undesirably resulted in irritation and red marking of the skin of the wearer. The high forces have also resulted in an undesirably level of bunching of the disposable absorbent article especially in the crotch region which is not often considered as being aesthetically pleasing.

Despite these and other attempts to develop more aesthetically pleasing and improved disposable absorbent articles, there remains a need for articles which contain body exudates while providing an optimum fit about the wearer and maintaining a pleasing appearance even after becoming saturated. Moreover there remains a need for disposable absorbent articles which have a garment-like appearance and which can be easily secured about and removed from the waist of a wearer.

### SUMMARY

In response to the foregoing need, the present inventors undertook intensive research and development efforts that resulted in the discovery of an improved dual fastening system for use with disposable absorbent articles. In one version, a disposable absorbent article is provided. The disposable absorbent article has opposed longitudinal side edges, opposed lateral end edges, a front waist region, a rear waist region and a crotch region. The crotch region extends between and connects the front waist region and the rear waist region. The front waist region has a width extending between the opposed longitudinal side edges. The rear waist region has a width extending between opposed longitudinal side edges. The disposable absorbent article also includes an outer cover, a liner, an absorbent core and a dual fastening system. The outer cover comprises a stretchable material having a bodyfacing surface and a garment facing surface. The liner is superposed over the bodyfacing surface of the outer cover. In addition, the liner comprises a stretchable material having a bodyfacing surface and a garment facing surface. The absorbent core is disposed between the garment facing surface of the liner and the bodyfacing surface of the outer cover. The dual fastening system comprises at least two primary fasteners and at least two secondary fasteners. At least a portion of each primary fastener is situated inboard from each longitudinal side edge of the rear waist region. Moreover, each primary fastener is configured to engage at least a portion of the garment facing surface of the outer cover. At least a portion of each secondary fastener is located in the front waist region and situated inboard from each longitudinal side edge of the front waist region. Each secondary fastener is configured to engage at least a portion of the bodyfacing surface of the liner. Also in this version, it is worth noting that the width of the front waist region is at least about 90 percent of the width of the rear waist region.

In another version, a disposable absorbent article is provided. The disposable absorbent article has opposed longitudinal side edges, opposed lateral end edges, a front waist region, a rear waist region and a crotch region. The crotch region extends between and connects the front waist region and the rear waist region. The front waist region has a width extending between the opposed longitudinal side edges. The rear waist region has a width extending between opposed longitudinal side edges. The disposable absorbent article also includes an outer cover, a liner, an absorbent core and a dual fastening system. The outer cover comprises a stretchable material having a bodyfacing surface and a garment facing surface. The liner is superposed over the bodyfacing surface of the outer cover. In addition, the liner comprises a stretchable material having a bodyfacing surface and a garment facing surface. The absorbent core is disposed between the garment facing surface of the liner and the bodyfacing surface of the outer cover. The dual fastening system comprises a pair of primary ears joined to the rear waist region. Each ear has a bodyfacing surface, a garment facing surface, a proximal edge, a distal edge, a first connecting edge and a second connecting edge. The first and second connecting edges connect the proximal and distal edges. Each ear also has a primary fastener. At least a portion of the primary fastener is situated inboard from the distal edge of the ear. Moreover, the primary fastener is configured to engage at least a portion of the garment facing surface of the outer cover. The dual fastening system also comprises a pair of secondary ears joined to the front waist region. Each ear has a bodyfacing surface, a garment facing surface, a proximal edge, a distal edge, a first connecting edge and a second connecting edge. The first and second connecting edges connect the proximal and distal edges. Each ear also has a secondary fastener. At least a portion of the secondary fastener is situated inboard from the distal edge of the ear. Moreover, the secondary fastener is configured to engage at least a portion of the bodyfacing surface of the liner. Also in this version, it is worth noting that the width of the front waist region is at least about 90 percent of the width of the rear waist region.

In yet another version, a disposable absorbent article is provided. The disposable absorbent article has opposed longitudinal side edges, opposed lateral end edges, a front waist region, a rear waist region and a crotch region. The crotch region extends between and connects the front waist region and the rear waist region. The front waist region has a width extending between the opposed longitudinal side edges. The rear waist region has a width extending between opposed longitudinal side edges. The disposable absorbent article also includes an outer cover, a liner, an absorbent core and a dual fastening system. The outer cover comprises an elastomeric material having a bodyfacing surface and a garment facing surface. The liner is superposed over the bodyfacing surface of the outer cover. In addition, the liner comprises an extensible material having a bodyfacing surface and a garment facing surface. The absorbent core is disposed between the garment facing surface of the liner and the bodyfacing surface of the outer cover. The dual fastening system comprises a pair of primary ears joined to the rear waist region. Each primary ear is made of a non-elastomeric material. Each ear has a bodyfacing surface, a garment facing surface, a proximal edge, a distal edge, a first connecting edge and a second connecting edge. The first and second connecting edges connect the proximal and distal edges. Each ear also has a primary fastener. At least a portion of the primary fastener is situated inboard from the distal edge of the ear. Moreover, the primary fastener is configured to engage at least a portion of the garment facing surface of the outer cover. The dual fastening system also comprises a pair of secondary ears joined to the front waist region. Each secondary ear is made of a non-elastomeric material. Each ear has a bodyfacing surface, a garment facing surface, a proximal edge, a distal edge, a first connecting edge and a second connecting edge. The first and second connecting edges connect the proximal and distal edges. Each ear also has a secondary fastener. At least a portion of the secondary fastener is situated inboard from the distal edge of the ear. Moreover, the secondary fastener is configured to engage at least a portion of the bodyfacing surface of the liner. Also in this version, it is worth noting that the width of the front waist region is at least about 90 percent of the width of the rear waist region.

### DRAWINGS

The foregoing and other features and aspects of the present invention and the manner of attaining them will become more apparent, and the invention itself will be better understood by reference to the following description, appended claims and accompanying drawings, where:

FIG. 1 illustrates a plan view of the bodyfacing surface of a representative disposable absorbent article incorporating a version of the improved dual fastening system;

FIG. 2 illustrates a plan view of the garment facing surface of a representative disposable absorbent article incorporating a version of the improved dual fastening system;

FIG. 3 illustrates a plan view of the bodyfacing surface of a representative disposable absorbent article incorporating a version of the improved dual fastening system; and

FIG. 4 illustrates a version of an ear suitable for incorporation into a version of the improved dual fastening system.

### DESCRIPTION

The present invention concerns an improved dual fastening system for use with disposable absorbent articles. The disposable absorbent articles are adapted to be worn adjacent to the body of a wearer to absorb and contain various exudates discharged from the body. As used herein, the term “disposable” refers to articles which are intended to be discarded after a limited use and which are not intended to be laundered or otherwise restored for reuse. The disposable absorbent articles of the present invention will be described in terms of a disposable diaper which is adapted to be worn by infants about the lower torso. It is understood that the dual fastening system of the present invention is equally adaptable for use with other types of disposable absorbent articles such as adult incontinent garments, children's training pants, and the like.

With regard to the designated surfaces of a disposable absorbent article and its components, the various upper or bodyfacing surfaces are configured to face toward the body of the wearer when the disposable absorbent article is worn by the wearer for ordinary use. The various opposing, lower or garment facing surfaces are configured to face away from the wearer's body when the disposable absorbent article is worn by the wearer.

As used herein, reference to two materials or elements being “joined” is intended to refer to the situation wherein the two materials or elements are directly joined to one another or where they are indirectly joined to one another or where they are indirectly joined to an intermediate element. Similarly, methods of joining two materials or elements include forming the elements or materials integrally, or attaching the elements together such as through the use of adhesive bonds, sonic bonds, thermal bonds, pinning, stitching, or a variety of other attachment techniques known in the art, as well as combinations thereof.

Stretchable materials may include materials that are extensible and materials that are elastomeric. Extensible materials typically have lower capacities to retract to their original lengths after stretching, while elastomeric materials typically have a greater range of stretch and come close to completely retracting to their original lengths.

It should be noted that the elongation, extension or permanent deformation properties of an extensible material are determined when the material is dry. Additionally, the percentage of elongation, extension or permanent deformation can be determined in accordance with the following formula:
100*[(L−Lₒ)/(Lₒ)]
where: L=elongated length; and
Lₒ=initial length.

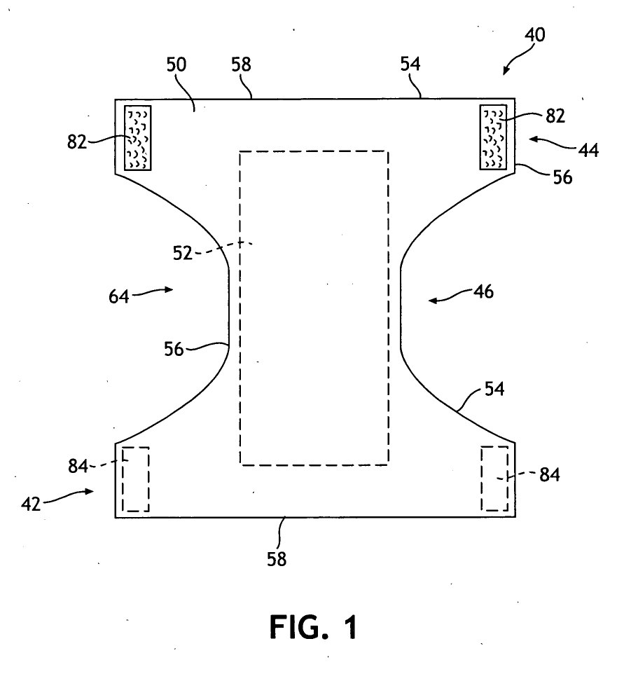

FIGS. 1-3 representatively illustrate examples of a disposable absorbent article, in this instance a diaper (as generally indicated at 40). Referring to FIGS. 1-3, the diaper (40) defines a front waist region (42), a rear waist region (44) and a crotch region (46) which extends between and connects the front (42) and rear (44) waist regions. The front waist region (42) comprises the portion of the diaper (40) which, when worn, is positioned on the front of the wearer, while the rear waist region (44) comprises the portion of the diaper which, when worn, is positioned on the back of the wearer. The crotch region (46) of the diaper (40) comprises the portion of the diaper which, when worn, is positioned between the legs of the wearer and covers the lower torso of the wearer.

The diaper (40), examples of which are illustrated in FIGS. 1-3, also includes an outer cover (48), a bodyside liner (50), and an absorbent core (52) situated between the outer cover (48) and the liner (50). The outer edges of the diaper (40) define a periphery (54) with laterally opposed, longitudinally extending side edges (56) and longitudinally opposed, laterally extending end edges (58). The diaper (40) may also include a system of elastomeric gathering members, such as leg elastics and waist elastics (not illustrated). The longitudinal side edges (56) define leg openings (64) for the diaper (40), and optionally are curvilinear and contoured. The lateral end edges (58) are illustrated as straight, but optionally, may be curvilinear. The diaper (40) additionally has a longitudinal centerline (66) and a lateral centerline (68). The diaper (40) may also include additional components to assist in the acquisition, distribution and storage of bodily waste. For example, the diaper (40) may include a transport layer, such as described in U.S. Pat. No. 4,798,603, issued to Meyer et al. (attorney docket number 8,263), or a surge management layer, such as described in European Patent Application Publication No. 0 539 703 (attorney docket number 9,922), published May 5, 1993.

As used herein, the term “inboard” is intended to refer to the direction from an edge toward a respective centerline. The term “outboard” is intended to refer to a direction away from a respective centerline.

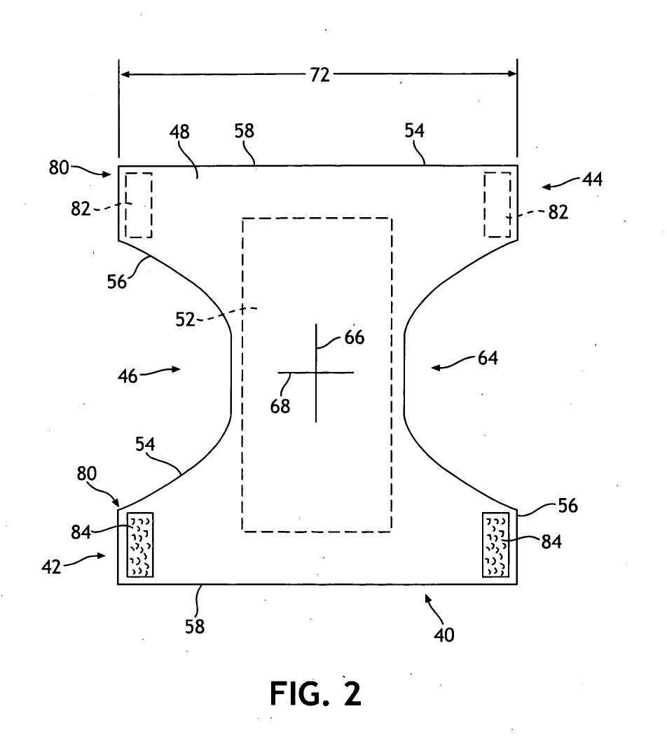

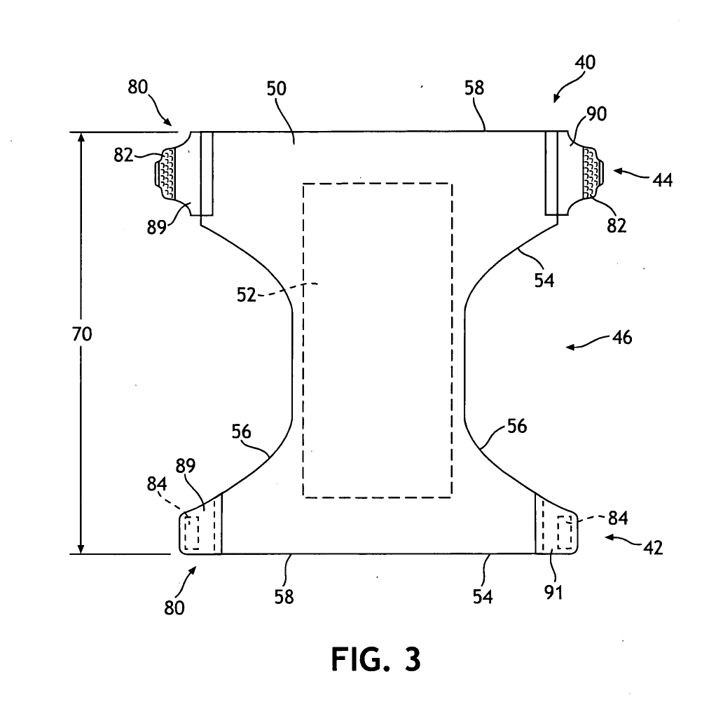

The diaper (40) generally defines a longitudinally extending length dimension (70), and a laterally extending width dimension (72) (as representatively illustrated in FIGS. 2 and 3). The diaper may have any desired shape, such as rectangular, I-shaped, a generally hourglass shape, or a T-shape.

The outer cover (48) and the liner (50) may be generally coextensive (e.g., FIG. 2), or optionally, may be non-coextensive. Either or both of the outer cover (48) and the liner (50) may have length and width dimensions which are generally larger than those of the absorbent core (52) and extend beyond the corresponding dimensions of the absorbent core (52) to provide longitudinal side edges (56) and lateral end edges (58) which may be connected or otherwise associated together in an operable manner.

Desirably the outer cover (48) is made of a stretchable material. In a particular aspect, the outer cover (48) is made of an elastomeric material. Suitable elastomeric materials are stretchable in one or more directions. Elastomeric materials may include cast or blown films, foams, or meltblown fabrics composed of polyethylene, polypropylene, or polyolefin copolymers, as well as combinations thereof. The elastomeric materials may include PEBAX elastomer (available from AtoChem located in Philadelphia, Pa.), HYTREL elastomeric polyester (available from E.I. DuPont de Nemours of Wilmington, Del.), KRATON elastomer (available from Kraton Polymers of Houston, Tex.), or strands of LYCRA elastomer (available from E.I. DuPont de Nemours of Wilmington, Del.), or the like, as well as combinations thereof. The outer cover (48) may include materials that have elastomeric properties through a mechanical process, printing process, heating process, or chemical treatment. For example, such materials may be apertured, creped, neck-stretched, heat activated, embossed, and micro-strained; and may be in the form of films, webs, and laminates.

In particular aspects, the outer cover (48) may include a 0.4 ounces per square yard (osy) (13.6 grams per square meter (gsm)) basis weight layer of G2760 KRATON elastomer strands adhesively laminated with a 0.3 gsm layer of adhesive between two facings. Each facing can be composed of a thermal point bonded bicomponent spunbond non-woven fibrous web having a 0.7 osy (23.7 gsm) basis weight. The adhesive is similar to an adhesive which is supplied by AtoFindley Adhesive, a business having offices in Wauwatosa, Wis., and designated as H2525A, and the elastomer strands are placed and distributed to provide approximately 12 strands of KRATON elastomer per inch (2.54 cm) of lateral width of the outer cover (48).

Materials suitable for a biaxially stretchable outer cover (48) include biaxially stretchable materials and biaxially elastic stretchable materials. One example of a suitable outer cover material can include a 0.3 osy polypropylene spunbond that is necked 60% in the lateral direction and creped 60% in the longitudinal direction, laminated with 3 grams per square meter (gsm) AtoFindley Adhesives H2525A styrene-isoprene-styrene based adhesive to 8 gsm PEBAX 2533 film with 20% TiO₂ concentrate. The outer cover (48) can suitably be stretched, laterally and/or longitudinally, by at least 30% (to at least 130% of an initial (unstretched) width and/or length of the outer cover (48)). More suitably, the outer cover (48) can be stretched laterally and/or longitudinally, by at least 50% (to at least 150% of the unstretched width or length of the outer cover (48)). Even more suitably, the outer cover (48) can be stretched, laterally and/or longitudinally, by at least 100% (to at least 200% of the unstretched width or length of the outer cover (48)). Tension force in the outer cover (48) at 50% extension is suitably between 50 and 1000 grams, more suitably between 100 and 600 grams, as measured on a 3 inch (7.62 cm) wide piece of the outer cover material.

Another example of a suitable material for a biaxially stretchable outer cover (48) is a breathable elastic film/nonwoven laminate, described in U.S. Pat. No. 5,883,028, issued to Morman et al. (attorney docket number 10,006), incorporated herein by reference to the extent that it is consistent (i.e., not in conflict) herewith. Examples of materials having two-way stretchability and retractability are disclosed in U.S. Pat. No. 5,116,662, issued to Morman (attorney docket number 8,801), and U.S. Pat. No. 5,114,781, issued to Morman (attorney docket number 9,585), both of which are hereby incorporated herein by reference to the extent that each is consistent (i.e., not in conflict) herewith. These two patents describe composite elastic materials capable of stretching in at least two directions. The materials have at least one. elastic sheet and at least one necked material, or reversibly necked material, joined to the elastic sheet at least at three locations arranged in a nonlinear configuration, so that the necked, or reversibly necked, web is gathered between at least two of those locations.

In an alternative aspect, the outer cover (48) is made of an extensible material. Extensible materials suitable for use as an outer cover (48) can provide an elongation of at least 10; alternatively, at least 20; alternatively, at least 30; or, alternatively, at least 40% when subjected to a tensile force of 30 gmf per inch (per 2.54 cm). Material suitable for use as an outer cover (48) can also provide a substantially permanent deformation of at least 10; alternatively, at least 15; alternatively, at least 20; alternatively, at least 25; or, alternatively, at least 30% when subjected to a tensile force of 50 gmf per inch (per 2.54 cm) and then allowed to relax, after removal of the tensile force, for a period of 1 minute. It should be readily appreciated that the described removal of the applied force results in a zero applied tensile stress and a zero applied tensile force.

The outer cover (48) can be composed of various materials and is suitably liquid impermeable. If extensible, for example, the outer cover (48) can be composed of a necked fabric, a creped fabric, a crimped fiber fabric, an extendable fiber fabric, a bonded-carded fabric, a micro-pleated fabric, polymer films or the like, as well as combinations thereof. The fabrics may be woven or non-woven materials, such as spunbond fabrics. In a particular aspect, the outer cover (48) can be composed of an extensible laminate of two or more layers. For example, the outer cover (48) may be a necked laminate formed from at least one neckable fabric laminated to at least one extendable film material wherein the necked laminate is extensible in at least one direction. The outer cover material (48), if extensible, may otherwise be a laminate formed from at least one necked fabric laminated to at least one extendable film material. In such a configuration, the laminate need not be necked. For purposes of the present description, the term “nonwoven web” refers to a web of fibrous material that is formed without the aid of a textile weaving or knitting process. The term “fabrics” is used to refer to woven, knitted and nonwoven fibrous webs. An example of an extensible material suitable for use as an outer cover (48) is a 60% necked, polypropylene spunbond having a basis weight of about 1.2 osy.

The liner (50) suitably presents a bodyfacing surface which is compliant, soft feeling, and non-irritating to the wearer's skin. Further, the liner (50) may be less hydrophilic than the absorbent core (52), to present a relatively dry surface to the wearer, and is sufficiently porous to be liquid permeable, permitting liquid to readily penetrate through its thickness. A suitable liner (50) may be manufactured from a wide selection of web materials, such as porous foams, reticulated foams, apertured plastic films, natural fibers (for example, polyester or polypropylene fibers), or a combination of natural and synthetic fibers. The liner (50) is suitably employed to help isolate the wearer's skin from liquids held in the absorbent core (50).

Desirably the liner (50) is made of a stretchable material. In a particular aspect, the liner (50) is made of an elastomeric material. Suitable elastomeric materials are stretchable in one or more directions. Suitable elastomeric materials for construction of the liner (50) can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from B.F. Goodrich and Company of Cleveland, Ohio), or PEBAX elastomers.

As an additional example, in one aspect the liner (50) suitably includes a non-woven, spunbond polypropylene fabric composed of about 2 to 3 denier fibers formed into a web having a basis weight of about 12 gsm which is necked approximately 60%. Strands of about 9 gsm KRATON G2760 elastomer material placed eight strands per inch (2.54 cm) are adhered to the necked spunbond material. The fabric is surface treated with an operative amount of surfactant, such as about 0.6 percent AHCOVEL Base N62 surfactant, available from ICI Americas, a business having offices in Wilmington, Del. The surfactant can be applied by any conventional means, such as spraying, printing, brush coating or the like. Other suitable materials may be extensible biaxially stretchable materials, such as a neck stretched/creped spunbond.

In an alternative aspect, the liner (50) is made of an extensible material. Extensible liner materials (50) can provide an elongation of at least 50% when subjected to a tensile force of 10 gmf per inch (per 2.54 cm). Extensible materials suitable for use as a liner (50) can also provide a substantially permanent deformation of at least 10% when subjected to a tensile force of 50 gmf per inch (per 2.54 cm) and then allowed to relax, after removal of the tensile force, for a period of 1 minute. It should be readily appreciated that the described removal of the applied force results in a zero applied tensile stress and a zero applied tensile force.

A suitable extensible liner (50) may be manufactured from a wide range of materials including, but not limited to woven and nonwoven materials, apertured formed thermoplastic films, apertured plastic films, hydro-formed films, porous foams, reticulated foams, reticulated thermoplastic films, and thermoplastic scrims. Suitable woven and nonwoven materials can include natural fibers (e.g., wood or cotton fibers), synthetic or modified natural fibers (e.g., polymeric fibers, such as polyester, polypropylene fibers, and polyethylene, or polyvinylalcohol, starch base resins, polyurethanes, cellulose esters, nylon, and rayon fibers), or a combination of natural and synthetic fibers. When the extensible liner material (50) includes a nonwoven web, the web may be spunbonded, carded, wet-laid, meltblown, hydroentangled, combinations of the above, or the like. An example of a suitable extensible liner (50) is a 50% necked, polypropylene spunbond having a basis weight of about 0.5 osy.

There are several known methods of providing a material above-described with extensibility. A non-limiting number of these methods are described herein. One such method would be to have the material made extensible by forming it from extensible or stretchable materials. Another example would be to mechanically manipulate a suitable material suitable. One such example of mechanical manipulation is described in U.S. Pat. No. 4,965,122, issued to Mormon (attorney docket number 8,730), the entire disclosure of which is incorporated herein by reference in a manner that is consistent (i.e., not in conflict) herewith. Examples of suitable manufacturing techniques are also described in U.S. Pat. No. 4,965,122, issued to Mormon.

For purposes of the present discussion, the term “% necked” or “percent neckdown” is intended to refer to a ratio or percentage determined by measuring the difference between the pre-necked dimension and the necked dimension of a neckable material, and then dividing that difference by the pre-necked dimension of the neckable material. The percentage of necking (percent neck) can be determined in accordance with the description in the above-mentioned U.S. Pat. No. 4,965,122, issued to Mormon.

As previously mentioned, the liner material (32) may be treated with a surfactant. This can be accomplished by a variety of techniques known to those skilled in the art. Treating the liner material (32) with a surfactant generally renders the liner material (32) more hydrophilic. This typically results in liquid penetrating the liner material (32) faster than if it were not treated.

The absorbent core (52) may include a matrix of hydrophilic fibers, such as a web of cellulosic fluff, mixed with particles of a high-absorbency material commonly known as superabsorbent material. In a particular version, the absorbent core (50) includes a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed.

The absorbent core (52) may have any of a number of shapes. For example, the absorbent core (52) may be rectangular, I-shaped or T-shaped. It is often desired that the absorbent core (52) be narrower in the crotch portion than the rear or front portion(s).

The high-absorbency material can be selected from natural, synthetic and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble, but swellable. Such means can include, for example, physical entanglement, crystalline domains, covalent bonds, ionic complexes and associations, hydrophilic associations, such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat, No. 4,286,082, issued to Tsubakimoto et al.

The high-absorbency material may be in a variety of geometric forms. It is desired that the high-absorbency material be in the form of discrete particles. However, the high-absorbency material may also be in the form of fibers, flakes, rods, spheres, needles, or the like. Often, the high-absorbency material is present in the absorbent core (50) in an amount of from about 5 to about 100 weight percent based on total weight of the absorbent core (50).

Referring again to FIGS. 1-3, illustrated are versions of a diaper (40) in its generally flat-out, uncontracted state (i.e., with all elastic induced gathering and contraction removed). The diaper (40) includes a liner (50) and an outer cover (48) which are coextensive and have length and width dimensions generally larger than those of an absorbent core (52). The liner (50) is associated with and superposed on the outer cover (48) to thereby form the periphery (54) of the diaper (40). The periphery (54) defines an outer perimeter or edge(s) of the diaper (40). The periphery (54) generally includes longitudinal side edges (56) and lateral end edges (58).

The disposable absorbent articles described herein also include a dual fastening system (80) for securing the absorbent article about the waist of the wearer. The illustrated versions of the diaper (40) include such a dual fastening system (80). Specifically, the dual fastening system includes a pair of primary fasteners (82) and a pair of secondary fasteners (84). In at least one version, the primary fasteners (82) are situated in the rear waist region (44) of the diaper (40), and located inboard each longitudinal side edge (56). The primary fasteners (82) are configured to encircle the hips of the wearer and engage the outer cover (48) of the front waist region (42) of the diaper (40) for holding the diaper on the wearer. Desirably, the primary fasteners (82) are releasably engageable directly with the garment facing surface of the outer cover (48). Alternatively, the diaper (40) may include a fastening panel (not illustrated) situated in the front waist region (42) of the garment facing surface of the outer cover (48). In such a configuration, the primary fasteners (82) are releasably engageable with the fastening panel to maintain the diaper (40) about the waist of the wearer. As representatively illustrated in FIGS. 1 and 3, the primary fasteners (82) may be hook type fasteners and the outer cover (48) or fastening panel may be configured to function as a complimentary loop type fastener. Desirably, the primary fasteners (82) are hook type fasteners which are releasably engageable with the outer cover (48). Such an arrangement provides the ability to vary the size of the waist opening in very small increments over a wide range to fit the waist of the wearer. The primary fasteners (82) may have a variety of shapes and sizes which provide the desired fastening of the diaper (40) about the waist of the wearer.

The dual fastening system (80) of the present invention further includes a pair of secondary fasteners (84) to provide improved securement of the diaper (40) about the waist of the wearer. For example, as representatively illustrated in FIGS. 1-3, the diaper (40) may include a pair of secondary fasteners (84) situated in the front waist region (42) of the diaper, and located adjacent each longitudinal side edge (56). The secondary fasteners (84) are configured to encircle the hips of the wearer and engage the bodyfacing surface of the liner (50) in the rear waist region (44) of the diaper (40). Suitably, as representatively illustrated in FIG. 2, the secondary fasteners (84) are hook type fasteners which are releasably engageable directly with the bodyfacing surface of the liner (50). Alternatively, the diaper (40) may include one or more fastening panels (not illustrated) on the bodyfacing surface of the rear waist region (44) to which the secondary fasteners (84) are releasably engageable.

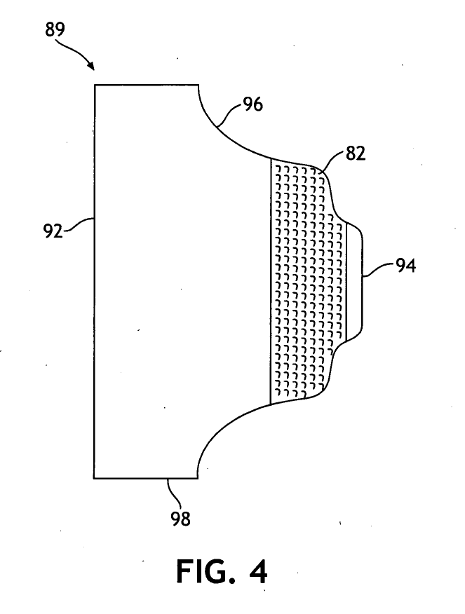

An alternative dual fastening system (80) may include one or more ears (89). As illustrated in FIGS. 3 and 4, each ear (89) includes a proximal edge (92), an opposed distal edge (94), a first connecting edge (96), and a second connecting edge (98). As used herein, the proximal edge (92) is that edge of the ear (89) which is joined to a longitudinal side edge (56) of the diaper (40). The distal edge (94) is that edge of the ear (89) which is opposite the proximal edge (92) moving in a direction outboard from a longitudinal centerline (66) of the diaper (40). The first (96) and second (98) connecting edges connect the proximal edge (92) and the distal edge (94) thereby defining a body of material which at least partially defines an ear (89). The ear (89) may be made of a variety of materials including those that are extensible, elastomeric and/or non-elastomeric. Desirably, the ear (89) is made of a non-elastomeric material.

The diaper (40) illustrated in FIG. 3 includes a version of the alternative dual fastening system (80) described herein. Specifically, the dual fastening system (80) includes a pair of primary fasteners (82) and a pair of secondary fasteners (84). Typically, the primary fasteners (82) are located inboard of a distal edge (94) on a bodyfacing surface of a primary ear (90). At least a portion of the proximal edge (92) of the primary ear (90) is joined to the rear waist region (44) of the diaper (40), with the distal edge (94) of the ear extending outboard from the longitudinal side edge (56) of the diaper. Desirably, the primary fasteners (82) are releasably engageable directly with the garment facing surface of the outer cover (48). Alternatively, the diaper (40) may include a fastening panel (not illustrated) situated in the front waist region (42) of the garment facing surface of the outer cover (48). In such a configuration, the primary fasteners (82) are releasably engageable with the fastening panel to maintain the diaper (40) about the waist of the wearer.

The dual fastening system of this alternate version also includes a pair of secondary fasteners (84). Specifically, the secondary fasteners (84) are located inboard of a distal edge (94) on a garment facing surface of a secondary ear (91). At least a portion o f the proximal edge (92) of the secondary ear (91) is joined to the front waist region (42) of the diaper (40), with the distal edge (94) of the ear extending outboard from the longitudinal side edge (56) of the diaper. Desirably, the secondary fasteners (84) are hook type fasteners which are releasably engageable directly with the bodyfacing surface of the liner (50). Alternatively, the diaper (40) may include one or more fastening panels (not illustrated) on the bodyfacing surface of the rear waist region (44) to which the secondary fasteners (84) are releasably engageable.

Suitable fasteners are well known to those of skill in the art and can include adhesive tape tab fasteners, hook and loop fasteners, mushroom fasteners, snaps, pin, belts and the like, and combinations thereof. For example, as representatively illustrated in FIGS. 1 and 3, the primary fasteners (82) may be hook type fasteners and the outer cover (48) or fastening panel (not illustrated) may be configured to function as a complimentary loop type fastener. Desirably, the primary fasteners (82) are hook type fasteners which are releasably engageable With at least a portion of an outer cover (48). The secondary fasteners (84) may also be hook type fasteners (as representatively illustrated in FIG. 2) and the liner (50) or fastening panel (not illustrated) may be configured to function as a complimentary loop type fastener. Desirably, the secondary fasteners (84) are hook type fasteners which are releasably engageable with at least a portion of a liner (50). Such an arrangement provides the ability to vary the size of the waist opening in very small increments over a wide range to fit the waist of the wearer. The primary (82) and secondary (84) fasteners may have a variety of shapes and sizes which provide the desired fastening of the diaper about the waist of the wearer.

The present inventors have discovered that certain advantages are achieved by configuring a disposable absorbent article such as a diaper with the dual fastening system described herein. Specifically, when the dual fastening system disclosed herein is incorporated into disposable absorbent articles having stretchable materials used in both the outer cover (48) and the bodyside liner (50), the present inventors believe that an optimum fit about the wearer is achieved while maintaining a pleasing appearance even after becoming saturated. The forces generated by the various versions of the dual fastening system described herein (ie, forces between the outer cover and the primary fasteners, and the liner and the secondary secondary fasteners when the disposable absorbent article is being worn by the wearer) provide a more secure fit of the disposable absorbent article about the wearer. These advantages are believed to be optimized when (i) the outer cover (48) comprises an elastomeric material; (ii) the liner (50) comprises either an elastomeric material or an extensible material; and (iii) the width of the front waist region (42) is at least 90 percent of the width of the rear waist region (44).

One version of the present invention discloses a disposable absorbent article having a dual fastening system. The disposable absorbent article has opposed longitudinal side edges (56), opposed lateral end edges (58), a longitudinal centerline (66), a front waist region (42), a rear waist region (44) and a crotch region (46) which extends between and connects the front waist region and the rear waist region. The front waist region (42) has a width extending between opposed longitudinal side edges (56). The rear waist region (44) has a width extending between opposed longitudinal side edges (56). The width of the front waist region (42) is at least about 90; alternatively, at least 90; alternatively, at least about 100; and finally, alternatively, at least 100 percent of the width of the rear waist region (44). The absorbent article also includes an outer cover (48), a bodyside liner (50), an absorbent core (52), and a dual fastening system (80). The outer cover (48) has a bodyfacing surface and a garment facing surface. The outer cover (48) desirably comprises a stretchable material. More desirably, the outer cover (48) comprises an elastomeric material. The liner (50) has a bodyfacing surface and a garment facing surface, and is superposed over the bodyfacing surface of the outer cover (48). The liner (50) desirably comprises a stretchable material. More desirably, the liner (50) comprises an extensible material. The absorbent core (52) is disposed between the garment facing surface of the liner (50) and the bodyfacing surface of the outer cover (48). The dual fastening system (80) includes at least two primary fasteners (82) and two secondary fasteners (84). At least a portion of each primary fastener (82) is located in the rear waist region (44) and situated inboard from each longitudinal side edge (56) of the rear waist region. Desirably, at least a portion of a primary fastener (82) is situated inboard and adjacent a respective longitudinal side edge (56) in the rear waist region (44). Each of the primary fasteners (82) is configured to releasably engage at least a portion of the garment facing surface of the outer cover (48). At least a portion of each secondary fastener (84) is located in the front waist region (42) and situated inboard from each longitudinal side edge (56) of the front waist region. Desirably, at least a portion of a secondary fastener (84) is situated inboard and adjacent a respective longitudinal side edge (56) in the front waist region (42). Each of the secondary fasteners (84) is configured to releasably engage at least a portion of the bodyfacing surface of the liner (50).

Another version of the present invention discloses a disposable absorbent article having a dual fastening system. The disposable absorbent article has opposed longitudinal side edges (56), opposed lateral end edges (58), a longitudinal centerline (66), a front waist region (42), a rear waist region (44) and a crotch region (46) which extends between and connects the front waist region and the rear waist region. The front waist region (42) has a width extending between opposed longitudinal side edges (56). The rear waist region (44) has a width extending between opposed longitudinal side edges (56). The width of the front waist region (42) is at least about 90; alternatively, at least 90; alternatively, at least about 100; and finally, alternatively, at least 100 percent of the width of the rear waist region (44). The absorbent article also includes an outer cover (48), a bodyside liner (50), an absorbent core (52), and a dual fastening system (80). The outer cover (48) has a bodyfacing surface and a garment facing surface. The outer cover (48) desirably comprises a stretchable material. More desirably, the outer cover (48) comprises an elastomeric material. The liner (50) has a bodyfacing surface and a garment facing surface, and is superposed over the bodyfacing surface of the outer cover (48). The liner (50) desirably comprises a stretchable material. More desirably, the liner (50) comprises an extensible material. The absorbent core (52) is disposed between the garment facing surface of the liner (50) and the bodyfacing surface of the outer cover (48). The dual fastening system (80) includes at least two primary ears (90) joined to the rear waist region (44). Each primary ear (90) has a bodyfacing surface, a garment facing surface, a proximal edge (92), a distal edge (94), a first connecting edge (96), a second connecting edge (98) and a primary fastener (82). The first (96) and second (98) connecting edges connect the proximal (92) and distal (94) edges. At least a portion of each primary fastener (82) is situated inboard from the distal edge (94) of each primary ear (90). Moreover, each primary fastener (82) is configured to engage at least a portion of the garment facing surface of the outer cover (48). The dual fastening system also includes at least two secondary ears (91) joined to the front waist region (42). Each secondary ear (91) has a bodyfacing surface, a garment facing surface, a proximal edge (92), a distal edge (94), a first connecting edge (96), a second connecting edge (98) and a secondary fastener (84). The first (96) and second (98) connecting edges connect the proximal (92) and distal (94) edges. At least a portion of each secondary fastener (84) is situated inboard from the distal edge (94) of each secondary ear (91). Moreover, each secondary fastener (84) is configured to engage at least a portion of the bodyfacing surface of the liner (50).

Yet another version of the present invention discloses a disposable absorbent article having a dual fastening system. The disposable absorbent article has opposed longitudinal side edges (56), opposed lateral end edges (58), a longitudinal centerline (66), a front waist region (42), a rear waist region (44) and a crotch region (46) which extends between and connects the front waist region and the rear waist region. The front waist region (42) has a width extending between opposed longitudinal side edges (56). The rear waist region (44) has a width extending between opposed longitudinal side edges (56). The width of the front waist region (42) is at least 90 percent of the width of the rear waist region (44). The absorbent article also includes an outer cover (48), a bodyside liner (50), an absorbent core (52), and a dual fastening system (80). The outer cover (48) has a bodyfacing surface and a garment facing surface. The outer cover (48) comprises an elastomeric material. The liner (50) has a bodyfacing surface and a garment facing surface, and is superposed over the bodyfacing surface of the outer cover (48). The liner (50) comprises an extensible material. The absorbent core (52) is disposed between the garment facing surface of the liner (50) and the bodyfacing surface of the outer cover (48). The dual fastening system (80) includes at least two primary ears (90) joined to the rear waist region (44). Each of the primary ears (90) is made of a non-elastomeric material. Each primary ear (90) has a bodyfacing surface, a garment facing surface, a proximal edge (92), a distal edge (94), a first connecting edge (96), a second connecting edge (98) and a primary fastener (82). The first (96) and second (98) connecting edges connect the proximal (92) and distal (94) edges. At least a portion of each primary fastener (82) is situated inboard from the distal edge (94) of each primary ear (90). Moreover, each primary fastener (82) is configured to engage at least a portion of the garment facing surface of the outer cover (48). The dual fastening system also includes at least two secondary ears (91) joined to the front waist region (42). Each of the secondary ears (91) is made of a non-elastomeric material. Each secondary ear (91) has a bodyfacing surface, a garment facing surface, a proximal edge (92), a distal edge (94), a first connecting edge (96), a second connecting edge (98) and a secondary fastener (84). The first (96) and second (98) connecting edges connect the proximal (92) and distal (94) edges. At least a portion of each secondary fastener (84) is situated inboard from the distal edge (94) of each secondary ear (91). Moreover, each secondary fastener (84) is configured to engage at least a portion of the bodyfacing surface of the liner (50).

One of skill in the art will readily appreciate that the various primary (82) and secondary (84) fasteners described herein may be combined to arrive at a number of configurations not illustrated herein, yet quite suitable for use in dual fastening systems. For example, one dual fastening system may utilize the primary fasteners (82) illustrated in FIGS. 1 and 2 with the secondary ears (91) and the secondary fasteners (84) illustrated in FIG. 3. In another example, an alternative dual fastening system may utilize the primary ears (90) and the primary fasteners (82) illustrated in FIG. 3 with the secondary fasteners (84) illustrated in FIGS. 1 and 2.

Having described the invention in rather full detail, it will be readily apparent that various changes and modifications can be made without departing from the spirit of the invention. All of such changes and modifications are contemplated as being within the scope of the invention as defined by the appended claims and any equivalents thereto.

1. A disposable absorbent article having opposed longitudinal side edges, opposed lateral end edges, a front waist region, a rear waist region and a crotch region which extends between and connects the front waist region and the rear waist region, the front waist region having a width extending between opposed longitudinal side edges and the rear waist region having a width extending between opposed longitudinal side edges, the disposable absorbent article comprising:
an outer cover comprising a stretchable material and having a bodyfacing surface and a garment facing surface;
a liner superposed over the bodyfacing surface of the outer cover, the liner comprising a stretchable material and having a bodyfacing surface and a garment facing surface;
an absorbent core disposed between the garment facing surface of the liner and the bodyfacing surface of the outer cover; and
a dual fastening system, the dual fastening system comprising:
at least two primary fasteners, at least a portion of each primary fastener being situated inboard from each longitudinal side edge of the rear waist region, each primary fastener being configured to engage at least a portion of the garment facing surface of the outer cover; and
at least two secondary fasteners, at least a portion of each secondary fastener being located in the front waist region and situated inboard from each longitudinal side edge of the front waist region, each secondary fastener being configured to engage at least a portion of the bodyfacing surface of the liner; and
wherein the width of the front waist region is at least about 90 percent of the width of the rear waist region.

2. The disposable absorbent article of claim 1, wherein the outer cover is an elastomeric material.

3. The disposable absorbent article of claim 2, wherein the liner is an extensible material.

4. The disposable absorbent article of claim 3, wherein the width of the front waist region is at least 90 percent of the width of the rear waist region.

5. The disposable absorbent article of claim 2, wherein the liner material is an elastomeric material.

6. The disposable absorbent article of claim 5, wherein the width of the front waist region is at least 90 percent of the width of the rear waist region.

7. A disposable absorbent article having opposed longitudinal side edges, opposed lateral end edges, a front waist region, a rear waist region and a crotch region which extends between and connects the front waist region and the rear waist region, the front waist region having a width extending between opposed longitudinal side edges and the rear waist region having a width extending between opposed longitudinal side edges, the disposable absorbent article comprising:
an outer cover comprising a stretchable material and having a bodyfacing surface and a garment facing surface;
a liner superposed over the bodyfacing surface of the outer cover, the liner comprising a stretchable material and having a bodyfacing surface and a garment facing surface;
an absorbent core disposed between the garment facing surface of the liner and the bodyfacing surface of the outer cover; and
a dual fastening system, the dual fastening system comprising:
a pair of primary ears joined to the rear waist region, each primary ear having a bodyfacing surface, a garment facing surface, a proximal edge, a distal edge, a first connecting edge and a second connecting edge, the first and second connecting edges connecting the proximal and distal edges, and a primary fastener, at least a portion of the primary fastener being situated inboard from the distal edge, the primary fastener being configured to engage at least a portion of the garment facing surface of the outer cover; and
a pair of secondary ears joined to the front waist region, each secondary ear having a bodyfacing surface, a garment facing surface, a proximal edge, a distal edge, a first connecting edge and a second connecting edge, the first and second connecting edges connecting the proximal and distal edges, and a secondary fastener, at least a portion of the secondary fastener being situated inboard from the distal edge, the primary fastener being configured to engage at least a portion of the bodyfacing surface of the liner; and
wherein the width of the front waist region is at least about 90 percent of the width of the rear waist region.

8. The disposable absorbent article of claim 7, wherein the outer cover is an elastomeric material.

9. The disposable absorbent article of claim 8, wherein the liner is an extensible material.

10. The disposable absorbent article of claim 9, wherein each primary fastener is situated outboard from at least the longitudinal side edge of the rear waist region.

11. The disposable absorbent article of claim 10, wherein each secondary fastener is situated outboard from at least the longitudinal side edge of the front waist region.

12. The disposable absorbent article of claim 11, wherein the width of the front waist region is at least 90 percent of the width of the rear waist region.

13. The disposable absorbent article of claim 7, wherein each primary fastener is situated outboard from at least the longitudinal side edge of the rear waist region.

14. The disposable absorbent article of claim 13, wherein the liner is an extensible material.

15. The disposable absorbent article of claim 14, wherein the outer cover is an elastomeric material.

16. The disposable absorbent article of claim 7, wherein each secondary fastener is situated outboard from at least the longitudinal side edge of the front waist region.

17. The disposable absorbent article of claim 16, wherein the liner is an extensible material

18. The disposable absorbent article of claim 17, wherein the outer cover is an elastomeric material.

19. A disposable absorbent article having opposed longitudinal side edges, opposed lateral end edges, a front waist region, a rear waist region and a crotch region which extends between and connects the front waist region and the rear waist region, the front waist region having a width extending between opposed longitudinal side edges and the rear waist region having a width extending between opposed longitudinal side edges, the disposable absorbent article comprising:
an outer cover comprising an elastomeric material and having a bodyfacing surface and a garment facing surface;
a liner superposed over the bodyfacing surface of the outer cover, the liner comprising an extensible material and having a bodyfacing surface and a garment facing surface;
an absorbent core disposed between the garment facing surface of the liner and the bodyfacing surface of the outer cover; and
a dual fastening system, the dual fastening system comprising:
a pair of primary ears joined to the rear waist region, each primary ear being made of a non-elastomeric material and having a bodyfacing surface, a garment facing surface, a proximal edge, a distal edge, a first connecting edge and a second connecting edge, the first and second connecting edges connecting the proximal and distal edges, and a primary fastener, the primary fastener being situated inboard from the distal edge of the ear and outboard from at least the longitudinal side edge of the rear waist portion, the primary fastener being configured to engage at least a portion of the garment facing surface of the outer cover; and
a pair of secondary ears joined to the front waist region, each secondary ear being made of a non-elastomeric material and having a bodyfacing surface, a garment facing surface, a proximal edge, a distal edge, a first connecting edge and a second connecting edge, the first and second connecting edges connecting the proximal and distal edges, and a secondary fastener, the secondary fastener being situated inboard from the distal edge of the ear and outboard from at least the longitudinal side edge of the front waist portion, the secondary fastener being configured to engage at least a portion of the bodyfacing surface of the liner; and
wherein the width of the front waist region is at least 90 percent of the width of the rear waist region.
